(19) United States
(12) Patent Application Publication
(10) Pub. No.: US 2023/0363992 A1
(43) Pub. Date: Nov. 16, 2023

(54) SILICONE-FREE ANTIPERSPIRANT AND DEODORANT COMPOSITIONS

(71) Applicant: KDC/ONE DEVELOPMENT CORPORATION, INC., Longueuil (CA)

(72) Inventors: Sabine REINES, Bromont (CA); Caroline MONPOU TRUCHET, Cowansville (CA)

(21) Appl. No.: 18/028,256

(22) PCT Filed: Sep. 28, 2021
(86) PCT No.: PCT/IB2021/058863

§ 371 Date: Mar. 24, 2023

Related U.S. Application Data
(60) Provisional application No. 63/084,099, filed on Sep. 28, 2020.

(51) Int. Cl. A61K 8/28; A61K 8/02; A61Q 15/00; A61K 8/37; A61K 8/92; A61K 8/34; A61K 8/25; A61K 8/73; A61K 8/49

(52) U.S. Cl. CPC A61K 8/28; A61K 8/0229; A61Q 15/00; A61K 8/37; A61K 8/922; A61K 8/342; A61K 8/25; A61K 8/732; A61K 8/498; A61K 8/0216; A61K 2800/34; A61K 2800/87; A61K 2800/75

(57) ABSTRACT

Described herein are antiperspirant and deodorant compositions which are advantageously free of silicone. Particular embodiments concern silicone-free compositions comprising at least one emollient, at least one structuring agent, and at least one natural or synthetic wax, wherein the at least one emollient comprises isoamyl laurate. Embodiments also concern silicone-free antiperspirant and deodorant compositions comprising a plurality of emollients, the plurality of emollients comprising isoamyl laurate. Also described are methods for making these compositions and others. Also described are uses of the silicone-free compositions for manufacturing antiperspirant sticks or antiperspirant creams, and/or for controlling malodors and transpiration.

### FIELD OF THE INVENTION

The invention relates to the field of personal hygiene, and more particularly to antiperspirants and deodorants that are free of silicone, and methods for making the same.

### BACKGROUND OF THE INVENTION

Deodorant is one of the most important personal care products used every day. A deodorant is a product applied to the body to combat or protect against body odor. A subgroup of deodorants and antiperspirants, not only affects the odor of the body, they also reduce sweating by affecting sweat glands. Antiperspirant prevents sweating by reducing moisture on the skin where perspiration-causing bacteria thrive. Antiperspirants are typically applied to the underarms, but both deodorants and antiperspirants may also be used on feet and other body areas. Generally, the product composition of deodorants and antiperspirants is different and sometimes controlled by health or regulatory authorities to ensure safety and minimal efficacy for the desired application.

Consumers are constantly demanding more from their personal care products and are constantly trying to improve their user experience, while being careful of the environment. For instance, personal care products comprising silicone are becoming less and less desirable because of safety concerns. However, while there are a lot of deodorants and antiperspirants on the market comprised of silicone, there is a very limited choice of silicone-free products. There is an even more reduced number, if any, of silicone-free deodorants and antiperspirants that provide a user experience comparable to silicone-containing products.

Accordingly, there is a need for antiperspirants and deodorants compositions that are free of silicone.

There is also a need for antiperspirants and deodorants that do not contain silicone while providing a user experience comparable to silicone-containing products in terms of look, feel, efficacy, etc.

There is also a need for antiperspirant and deodorant compositions that comprise biodegradable ingredients and that are environmentally friendly.

There is also a need for methods of manufacturing such silicone-free antiperspirant and deodorant compositions.

The present invention addresses these needs and other needs as it will be apparent from the review of the disclosure and description of the features of the invention hereinafter.

### BRIEF SUMMARY OF THE INVENTION

According to one aspect, the invention relates to a silicone-free antiperspirant and/or deodorant composition, the composition comprising at least one emollient, at least one structuring agent, and at least one natural or synthetic wax, wherein said at least one emollient comprises isoamyl laurate.

According to another aspect, the invention relates to a silicone-free antiperspirant and/or deodorant composition, the composition comprising a plurality of emollients, the plurality of emollients comprising isoamyl laurate.

According to another aspect, the invention relates to a silicone-free antiperspirant and/or deodorant composition comprising at least one emollient, at least one structuring agent, and at least one natural or synthetic wax, wherein said at least one emollient is selected from the group consisting of C₁₂-C₁₅ alkyl benzoate, isopropyl myristate, dicaprylyl carbonate, oleyl erucate, hydrogenated ethylhexyl olivate, hydrogenated olive oil unsaponifiables, C₁₅-C₁₉ alkane and mixtures thereof.

According to one particular aspect, the invention relates to a silicone-free antiperspirant composition (e.g., a stick) comprising, or alternatively consisting of:
about 35% w/w to about 55% w/w of emollient(s);
about 16% w/w to about 20% w/w of structuring agent(s);
about 4% w/w to about 6% w/w of natural wax(es);
about 12% w/w to about 20% w/w of antiperspirant agent(s);
about 1.5% w/w to about 3% w/w of suspension agent(s);
about 0.05% w/w to about 0.5% w/w of antioxidant(s);
about 0% w/w to about 3% w/w of additive(s);
about 1.5% w/w to about 3% w/w wetness absorber(s); and
about 0% w/w to about 1.5% w/w fragrances.

According to another particular aspect, the invention relates to a silicone-free antiperspirant composition (e.g., a cream) comprising, or alternatively consisting of:
about 2% w/w to about 5% w/w of humectant(s)/moisturizer(s);
about 0.05% w/w to about 1.5% w/w of thickener(s) (e.g., biobased thickeners);
about 15% w/w to about 25% w/w of emollient(s);
about 1% w/w to about 3% w/w of natural wax(es);
about 2% w/w to about 4% w/w of structuring agent(s);
about 2% w/w to about 4% w/w of emulsifier(s);
about 0.01% w/w to about 0.1% w/w of antioxidant(s);
about 0.5% w/w to about 3% w/w wetness absorber(s);
about 12% w/w to about 25% w/w of antiperspirant agent(s);
about 0% w/w to about 3% w/w of additive(s);
about 0% w/w to about 2.5% w/w fragrance(s); and
solvent(s) (e.g., water) to complete to 100% w/w.

According to another particular aspect, the invention relates to a silicone-free antiperspirant composition (e.g., a cream) comprising, or alternatively consisting of:
about 3% w/w of humectant(s)/moisturizer(s)
about 20.35% w/w of emollient(s);
about 1% w/w of thickener(s) (e.g., biobased thickeners);
about 3% w/w of structuring agent(s);
about 1.7% w/w of natural wax(es);
about 19% w/w of antiperspirant agent(s);
about 0.05% w/w of antioxidant(s);
about 1.9% w/w of additive(s);
about 1.4% w/w wetness absorber(s);
about 1.5% w/w fragrance(s); and
about 44.1% w/w solvent (e.g., water).

According to another aspect, the invention relates to a silicone-free antiperspirant and/or deodorant composition comprising isoamyl laurate and a natural or synthetic wax selected from the group consisting of jojoba esters, hydrogenated castor oil, beeswax coconut wax, soy wax, ozokerite, ceresin and mixtures thereof.

According to another aspect, the invention relates to a silicone-free antiperspirant and/or deodorant composition comprising:
a) at least one emollient selected from the group consisting of C₁₂-C₁₅ alkyl benzoate, isopropyl myristate, dicaprylyl carbonate, oleyl erucate, hydrogenated ethylhexyl olivate, hydrogenated olive oil unsaponifiables, C₁₅-C₁₉ alkane and mixtures thereof; and
b) a natural or synthetic wax selected from the group consisting of jojoba esters, hydrogenated castor oil, beeswax, coconut wax, soy wax, ozokerite, ceresin and mixtures thereof.

Additional aspects relate to the use isoamyl laurate in thee manufacture of a silicone-free antiperspirant and/or deodorant stick or cream, and to a method of making a silicone-free antiperspirant and/or deodorant stick or cream, comprising preparing a silicone-free antiperspirant and/or deodorant composition comprising isoamyl laurate.

Additional aspects relate to the use of an emollient selected from the group consisting of C₁₂-C₁₅ alkyl benzoate, isopropyl myristate, dicaprylyl carbonate, oleyl erucate, hydrogenated ethylhexyl olivate, hydrogenated olive oil unsaponifiables, C₁₅-C₁₉ alkane and mixtures thereof, in the manufacture of a silicone-free antiperspirant and/or deodorant stick or cream. A related aspect concerns a method of making a silicone-free antiperspirant and/or deodorant stick or cream comprising preparing a silicone-free antiperspirant and/or deodorant composition comprising at least one emollient selected from the group consisting of isoamyl laurate C₁₂-C₁₅ alkyl benzoate, isopropyl myristate, dicaprylyl carbonate, oleyl erucate, hydrogenated ethylhexyl olivate, hydrogenated olive oil unsaponifiables, C₁₅-C₁₉ alkane and mixtures thereof.

According to one particular aspect, the invention relates a method of making a silicone-free antiperspirant composition, In one embodiment, the method comprises the steps of:
1) premixing at least one emollient under heating;
2) successively adding at least one structuring agent and a natural or synthetic wax under mixing until completely melted and dispersed;
3) cooling the mixture of step 2) while maintaining a liquid mixture;
4) adding and mixing at least one antiperspirant agent, at least one suspension agent and at least one wetness absorber to the cooled liquid mixture of step 3);
5) optionally adding one or more additives selected from an antimicrobial agent, an antioxidant, a soothing agent, an odor fighting agent at a temperature preserving integrity of the additive(s);
6) homogenizing to ensure a proper suspension and dispersion of all ingredients; 7) optionally adding and mixing a fragrance;
thereby obtaining a silicone-free antiperspirant composition.

In embodiments, to obtain a soft-solid sticks, the method further comprises the steps of:
8) pouring the liquid mixture into stick molds; and
9) refrigerating molded sticks for solidification.

In other embodiments, to obtain a cream, the method comprises the steps of:
1) in a first vessel, premixing water and humectant(s)/moisturizer(s) under heating;
2) adding slowly thickener(s) until complete mixing and dispersion;
3) in a second vessel, premixing emollient(s), natural vegetal wax(es), structuring agent(s), emulsifier(s) and antioxidant(s) under heating;
4) adding the premix from the second vessel (step 3) to the premix of the first vessel (step 2) under heating and mixing;
5) adding wetness absorber(s) under mixing, and homogenizing to ensure a complete emulsion;
6) cooling the mixture/emulsion of step 5;
7) adding antiperspirant agent(s) (e.g. solubilized beforehand in a solvent such as water);
8) adding optionally additive(s) and fragrance(s) into the mixture of step 7;
9) cooling the mixture under mixing until it reaches room temperature, thereby obtaining a cream.

Related aspects concern personal hygiene products, such as antiperspirant sticks and creams, which comprises a silicone-free composition as defined herein, or that are manufactured according to the methods described herein.

Another related aspect concerns the use of a silicone-free composition as defined herein, and/or with a silicone-free composition manufactured according to a method defined herein, and/or with an antiperspirant stick as defined in herein, and/or with an antiperspirant cream as defined in herein, for controlling at least one of malodors and transpiration.

Another related aspect concerns methods of controlling at least one of malodors and transpiration, comprising contacting a situs comprising a malodor and/or a situs that will become malodorous with a silicone-free composition as defined herein, with a silicone-free composition manufactured according to a method defined herein, with an antiperspirant stick as defined in herein, and/or with an antiperspirant cream as defined in herein.

Additional aspects, advantages and features of the present invention will become more apparent upon reading of the following non-restrictive description of preferred embodiments which are exemplary and should not be interpreted as limiting the scope of the invention.

### DETAILED DESCRIPTION OF EMBODIMENTS

In the following description of the embodiments, are by way of illustration of an example by which the invention may be practiced. It will be understood that other embodiments may be made without departing from the scope of the invention disclosed.

### Silicone-Free Antiperspirant and/or Deodorant Compositions Comprising an Emollient

The invention relates to silicone-free antiperspirant and/or deodorant compositions suitable for topical applications on human skin. In preferred embodiments, the composition is a silicone-free antiperspirant that comprises at least active antiperspirant compound.

According to one desirable aspect, the invention relates to an antiperspirant and/or deodorant composition that is silicone-free. As used herein the term “silicone-free” refers to the total absence (e.g. 100% free) of any silicone compound such as linear and volatile and non-volatiles silicones. Examples of silicones compounds that are absent from the composition of the invention include, but are not limited to, cyclomethicone (e.g. cyclopentasiloxane), dimethicone, dimethiconol, phenyltrimethicone, cyclohexasiloxane and mixtures thereof. In embodiments the term “silicone-free” or “substantially free of silicone” refers to a composition which does not comprise any silicone compound, or comprises less than 1% w/w (i.e. 99% silicone-free), or comprises less than 0.5% w/w (i.e. 99.5% silicone-free), or comprises less than 0.1% w/w (i.e. 99.9% silicone-free), or comprises less than 0.05% w/w (i.e. 99.95% silicone-free), or comprises less than 0.01% w/w (i.e. 99.99% silicone-free) or only trace amounts like less than 5 ppm, less than 1 ppm or below detections limits.

According to embodiments, the silicone-free antiperspirant and/or deodorant composition is “anhydrous”, i.e. it is substantially free of added water (i.e. water is not added as a stand-alone ingredient but some amounts may be present in aqueous ingredients). In embodiments the term “anhydrous” refers to a silicone-free antiperspirant and/or deodorant composition which comprises less than 5% w/w (i.e. 95% water-free), 1% w/w (i.e. 99% water-free), or less than 0.5% w/w (i.e. 99.5% water-free), or less than 0.1% w/w (i.e. 99.9% water-free).

According to a related aspect, the invention relates to the use of isoamyl laurate as an emollient in the manufacture of a silicone-free antiperspirant and/or deodorant composition.

According to a related aspect, the invention relates to the use of one or more emollient selected from the group consisting of C₁₂-C₁₅ alkyl benzoate, isopropyl myristate, dicaprylyl carbonate, and mixtures thereof.

According to a related aspect, the invention relates to the use of one or more emollient comprised of at least one emollient selected from the group consisting of oleyl erucate, hydrogenated ethylhexyl olivate, hydrogenated olive oil unsaponifiables, C₁₅-C₁₉ alkane and mixtures thereof.

Accordingly, there is provided a silicone-free antiperspirant and/or deodorant composition comprising a plurality of emollients, wherein at least one of the plurality of emollients is isoamyl laurate.

According to a related aspect, the silicone-free antiperspirant and/or deodorant composition comprises one or more emollient, wherein the emollient is an ester suitable for application on the human skin and also compatible for use in antiperspirant and/or deodorant compositions. In embodiments the ester is selected from isoamyl laurate, C₁₂-C₁₅ alkyl benzoate, diheptyl succinate, C₁₃-C₁₆ isoparaffin, heptyl undecylenate, C₉-C₁₂ alkane, C₁₅-C₁₉ alkane, diheptyl succinate, capryloyl glycerin/sebacic acid copolymer.

According to a related aspect, the invention relates to a silicone-free antiperspirant and/or deodorant composition comprising a plurality of emollients, wherein the plurality of emollients comprises at least one emollient selected from the group consisting of C₁₂-C₁₅ alkyl benzoate, isopropyl myristate, dicaprylyl carbonate, oleyl erucate, hydrogenated ethylhexyl olivate, hydrogenated olive oil unsaponifiables, C₁₅-C₁₉ alkane and mixtures thereof.

According to a related aspect, the invention relates to a silicone-free antiperspirant and/or deodorant composition comprising: (i) at least one emollient and (ii) at least one natural wax, wherein the emollient is selected from the group consisting of isoamyl laurate, C₁₂-C₁₅ alkyl benzoate, isopropyl myristate, dicaprylyl carbonate, oleyl erucate, hydrogenated ethylhexyl olivate, hydrogenated olive oil unsaponifiables, C₁₅-C₁₉ alkane and mixtures thereof, and wherein the natural wax is selected from the group consisting of jojoba esters, hydrogenated castor oil, beeswax, coconut wax, soy wax, ozokerite, ceresin and mixtures thereof.

“Isoamyl laurate” is a chemical compound that may be known by other chemical names including, but not limited to, isopentyl laurate, isopentyl dodecylate, isoamyl laurate, isopentyl laurate, isopentyl dodecanoate, isoamyl dodecanoate, 3-methylbutyl dodecanoate, dodecanoic acid 3-methylbutyl ester, lauric acid isopentyl ester, lauric acid isoamyl ester, iso-amyl n-dodecanoate, lauricacidisoamylester, and Qualix™ D5-R77. The present invention encompasses compounds with these other chemical names. In embodiments, the term refers particularly to a compound having the molecular formula C₁₇H₃₄O₂.

“C₁₂-C₁₅ Alkyl Benzoate” is a chemical compound that may be known by other chemical names including, but not limited to, Benzoic acid, C₁₂-C₁₅-alkyl esters, Finsolv TN™, CAS 68411-27-8, INCI 270-112-4. The present invention encompasses compounds with these other chemical names. In embodiments, the term refers particularly to a compound having the molecular formula (CH₂)₁₁₋₁₄C₈H₈O₂.

“Isopropyl myristate” is a chemical compound that may be known by other chemical names including, but not limited to (Propan-2-yl tetradecanoate) OR (Tetradecanoic acid, 1-methylethyl ester) OR (Myristic acid isopropyl ester), Tegosoft M™, OR CAS 110-27-0. The present invention encompasses compounds with these other chemical names. In embodiments, the term refers particularly to a compound having the molecular formula C₁₇H₃₄O₂.

“Dicaprylyl Carbonate” is a chemical compound that may be known by other chemical names including, but not limited to, Cetiol CC™ or CAS 1680-31-or INCI 434-850-2. The present invention encompasses compounds with these other chemical names. In embodiments, the term refers particularly to a compound having the molecular formula C₁₇H₃₄O₃.

“Oleyl erucate,” is a chemical compound that may be known by other chemical names including, but not limited to (Z)-Octadec-9-enyl (Z)-docos-13-enoate, Tegosoft OER™, CAS 17673-56-2, INCI 143485-69-2. The present invention encompasses compounds with these other chemical names. In embodiments, the term refers particularly to a compound having the molecular formula C₄H₇₆O₂.

“Hydrogenated Ethylhexyl Olivate, Hydrogenated Olive Oil Unsaponifiables” is a chemical compound that may be known by other chemical names including, but not limited to, octadecanoic acid, 2-ethylhexyl ester, Tetracosane, 2,6,10,15,19,23-hexamethyl), Fision EcoSil™, CAS 22047-49-0. The present invention encompasses compounds with these other chemical names. In embodiments, the term refers particularly to a compound having the molecular formula C₂₆H₅₂O₂.

“C₁₅-C₁₉ Alkane” is a chemical compound that may be known by other chemical names including, but not limited to, Emosmart L19™, Emosmart Green™ OR EmoGreen™ or CAS 64741-76-0 or CAS 64742-46-7 or INCI 265-077-7 OR INCI 265-148-2. The present invention encompasses compounds with these other chemical names.

Any suitable mixture of emollient(s) may be used in accordance with the present invention. In one embodiment, the mixture of emollient(s) comprises at least isoamyl laurate, such as the product commercialized under the brand name Qualix D5R-77™. Advantageously, this particular product is known to be derived from natural building blocks and is readily biodegradable. Its low viscosity and a quick absorption results in spreading and quick penetration properties and it provides an excellent silicone-like feeling and has good skin compatibility. It can also act as an anti-caking agent. A person skilled in the art can identify other suitable products with similar desirable properties. Therefore, the present invention encompasses additional emollients having similar properties to isoamyl laurate and Qualix D5R-77™.

In embodiments, the following emollient(s) is(are) used alone or in combination in order to substitute for isoamyl laurate: C₁₃-C₁₆ isoparaffin & heptyl undecylenate (e.g. Lexfeel wow DT™), C₉-C₁₂ alkane (e.g. Jeen NDA™) diheptyl succinate & capryloyl glycerin sebacic acid polymer (e.g. Lexfeel N5™). These emollients may also be mixed and combined with isoamyl laurate.

In one embodiment, the mixture of emollient(s) comprises C₁₂-C₁₅ alkyl benzoate such as the product commercialized under the brand name Finsolv TN™. Advantageously, this particular product confers a dry, talc-like feel and is extremely mild to skin and spreads easily (high spreading coefficient). A person skilled in the art can identify other suitable products with similar desirable properties. Therefore, the present invention encompasses additional emollients having similar properties to C₁₂-C₁₅ alkyl benzoate or Finsolv TN™. In embodiments, the following emollient(s) is(are) used alone or in combination in order to substitute for C₁₂-C₁₅ alkyl benzoate (or is used in addition thereto): Isopropyl myristate (e.g. Tegosoft M™) and/or Dicaprylyl Carbonate (e.g. Cetiol CC™). These emollients may also be added to C₁₂-C₁₅ alkyl benzoate.

In one embodiment, the mixture of emollient(s) comprises oleyl erucate, such as the product commercialized under the brand name Tegosoft OER™. Advantageously, this particular product imparts a caring and smooth skin feel. It is fully biodegradable and, when appropriate, it can be used as an alternative to natural jojoba oil. A person skilled in the art can identify other suitable products with similar desirable properties. Therefore, the present invention encompasses additional emollients having similar properties to oleyl erucate or Tegosoft OER™.

In one embodiment, the mixture of emollient(s) comprises hydrogenated ethylhexyl olivate, such as the product commercialized under the brand name Fision EcoSil™. Advantageously, this particular product is odorless and colorless. It assists in reducing greasiness and tackiness of the final composition. It is soft and light with a dry after-feel, such that the skin feels smooth and moisturized. It is natural and botanically derived and the nourishing olive supports a healthy skin. A person skilled in the art can identify other suitable products with similar desirable properties. Therefore, the present invention encompasses additional emollients having similar properties to hydrogenated ethylhexyl olivate, hydrogenated olive oil unsaponifiables or Fision EcoSil™.

In embodiments, the silicone-free antiperspirant and/or deodorant composition comprises about 20% w/w to about 55% w/w emollient(s), or about 40% w/w to about 50% w/w emollient(s), or about 45-50% w/w emollient(s).

According to embodiments, the silicone-free composition further comprises at least one of a structuring agent. The present invention encompasses the use of any structuring agent suitable for application on the human skin and also compatible for use in antiperspirant and/or deodorant compositions. In embodiments the structuring agent is selected from hydrogenated castor oils (e.g. Castorwax™ MP-80), cetyl alcohol, cetearyl alcohol (including, but not limited to, C₁₆-C₁₈ cetearyl alcohol), myristyl alcohol, stearyl alcohol (including, but not limited to, C₁₈ stearyl alcohol), and mixtures thereof. In embodiments, the silicone-free antiperspirant and/or deodorant composition comprises about 15% w/w to about 25% w/w structuring agent(s), or about 20% w/w to about 25% w/w structuring agent(s).

According to embodiments, the silicone-free composition further comprises at least one thickener, preferably biobased thickener(s). The present invention encompasses the use of any biobased thickener suitable for application on the human skin and also compatible for use in antiperspirant and/or deodorant compositions. The biobased thickener may also be selected to act as a gelifier, gelifying agent, texturizer, and/or emulsion stabilizer. In embodiments, the biobased thickener is selected from FiberDesign Sensation™ (Cargill). In embodiments, the silicone-free antiperspirant and/or deodorant composition comprises about 0.05% w/w to about 1.5% w/w thickener(s), or about 1% w/w thickener(s).

According to embodiments, the silicone-free composition further comprises at least one emulsifier. The present invention encompasses the use of any emulsifier suitable for application on the human skin and also compatible for use in antiperspirant and/or deodorant compositions. In embodiments the emulsifier is selected from Emulgin B2™ (Ceteareth-20). In embodiments, the silicone-free antiperspirant and/or deodorant composition comprises about 1% w/w to about 5% w/w, or about 2% w/w to about 4% w/w emulsifier(s), or about 3% w/w thickener(s).

According to embodiments, the silicone-free composition further comprises at least one of a natural or a synthetic wax. The present invention encompasses the use of any natural or synthetic wax suitable for applications on the human skin and also compatible for use in antiperspirant and/or deodorant compositions. In embodiments the wax is a natural wax, e.g. a white natural wax from vegetal or animal source such as jojoba esters (e.g. Floraesters 70™), hydrogenated castor oil (e.g. Castoroil MP-80™), beeswax, coconut wax, soy wax, and mixtures thereof. In other embodiments the natural wax is selected from candelilla wax, carnauba wax, Brazil wax, palm wax, beeswax, rice bran wax, Helianthus annuus (sunflower) seed wax, Acacia decurrens flower wax, Citrus aurantium dulcis (orange) peel wax, Rosa centifolia flower wax, and berry wax. In embodiments the wax is a synthetic wax from a mineral or synthetic source, including but not limited to ozokerite, ceresin, syncrowax, and mixtures thereof. A person skilled in the art can identify other suitable products with similar desirable properties. The present invention encompasses additional natural and synthetic waxes having similar properties to those particular products listed hereinabove. In embodiments, the silicone-free antiperspirant and/or deodorant composition comprises about 3% w/w to about 10% w/w natural and/or synthetic wax(es), or about 4% w/w to about 6% w/w natural and/or synthetic wax(es), or about 5% w/w to about 8% w/w natural and/or synthetic wax(es), or about 6% w/w natural and/or synthetic wax(es).

In embodiments, the silicone-free antiperspirant and/or deodorant composition comprises a combined amount of structuring agent+natural and/or synthetic wax(es) of about 20% w/w to about 30% w/w, or about 20% w/w to about 26% w/w, or about 23% w/w to about 28% w/w, or about 25% w/w to about 26% w/w.

According to embodiments the antiperspirant composition further comprises a suspension or cohesion agent. Such suspension agent is typically in a powder form and is preferable for assisting in the suspension of the various ingredients (e.g. antiperspirant agent(s)) and give a final antiperspirant composition in which the ingredients are united as a whole. Examples of envisioned suspension agents include, but are not limited to, silica (e.g. Cabosil M5P™), cellulose powder(s), mixtures thereof, etc. Preferably, the suspension agent is fully biodegradable and advantageously it imparts to the composition a soft matte finish. A person skilled in the art can identify other suitable products with similar desirable properties. Therefore, the present invention encompasses additional suspension agent having similar properties to silica and cellulose powder products listed hereinabove. In embodiments, the silicone-free antiperspirant and/or deodorant composition comprises about 1.5% w/w to about 3% w/w suspension or cohesion agent(s), or about 2.5% w/w suspension or cohesion agent(s).

According to embodiments, the silicone-free composition further comprises at least one wetness absorber. The present invention encompasses the use of any wetness absorber suitable for application on the human skin and also compatible for use in antiperspirant and/or deodorant compositions. In embodiments the wetness absorber is aluminum starch octenylsuccinate (e.g. Dry-Flo Pure™). In other embodiments the wetness absorber is selected from tapioca starch, potato starch, rice starch, corn starch, arrowroot powder, clays, hectorite, bentonite, zeolite and mixtures thereof. A person skilled in the art can identify other suitable products with similar desirable properties. Therefore, the present invention encompasses additional wetness absorber having similar properties to those products listed hereinabove. In embodiments, the silicone-free antiperspirant and/or deodorant composition comprises about 1.5% w/w to about 3% w/w wetness absorber(s) or about 2.5% w/w wetness absorber(s).

According to preferred embodiments the silicone-free composition is silicone-free antiperspirant composition and, accordingly, it further comprises an active antiperspirant agent. The present invention encompasses the use of any antiperspirant agent suitable for application on the human skin and also compatible for use in antiperspirant and/or deodorant compositions. In embodiments the antiperspirant agent is an aluminum salt. In embodiments the antiperspirant agent is selected from aluminium zirconium trichlorohydrex Gly (e.g. Achieve DA 5390-04™, Elementis AAZG-7165™), aluminium zirconium tetrachlorohydrex Gly, aluminum zirconium pentachlorohydrex Gly, aluminum zirconium octachlorohydrex Gly, aluminium chlorohydrate, and aluminum sesquichlorohydrate. In embodiments, the silicone-free antiperspirant and/or deodorant composition comprises about 12% w/w to about 20% w/w antiperspirant agent(s), or about 13% w/w to about 18% w/w antiperspirant agent(s), or about 15% w/w to about 16% w/w antiperspirant agent(s).

According to embodiments the silicone-free composition further comprises at least one of a fragrance and an essential oil. The present invention encompasses the use of any fragrance and/or essential oil suitable for application on the human skin and also compatible for use in antiperspirant and/or deodorant compositions. The fragrance and/or essential oil may be particularly useful for masking odors and also create a product signature. In embodiments, the silicone-free antiperspirant and/or deodorant composition comprises about 0% w/w to about 3% w/w fragrance(s) and essential oil(s), or about 0.5% w/w to about 2.5%, about 1% w/w to about 1.5% fragrance(s) and essential oil(s).

According to embodiments, the silicone-free composition further comprise at least one antioxidant suitable for application on the human skin and also compatible for use in antiperspirant and/or deodorant compositions. Antioxidant(s) are preferable for protecting the emollient(s) from oxidation. Suitable antioxidants include, but are not limited to, tocopherol, tocopheryl actetate (e.g. Vitamin E Acetate Care™), and Pentaerythrityl Tetra-Di-t-Butyl Hydroxyhydrocinnamate (e.g. Tinogard TT™). In embodiments, the silicone-free antiperspirant and/or deodorant composition comprises about 0.05% w/w to about 0.5% antioxidant(s), or about 0.1% w/w to about 0.25% antioxidant(s).

The silicone-free composition may further comprise one or more additives including, but not limited to, at least one of an antimicrobial agent, a soothing agent, an anti-inflammatory agent (e.g., Tazman Pepper™) and an aesthetic agent (e.g. mica, titanium dioxide) that is suitable for application on the human skin and also compatible for use in antiperspirant and/or deodorant compositions. One particular example of an antimicrobial agent, soothing and/or anti-inflammatory agent is a Zingiber officinalis (Ginger) root extract (e.g. Symrelief™ commercialized by Symrise). These ingredients are optional and in embodiments, any of the antimicrobial agent, soothing agent, anti-inflammatory agent and esthetic agent may be present in about 0% w/w w/w to about 3% w/w, or about 0.05% w/w w/w to about 3% w/w, or about 0.1% w/w to about 3%, or about 0.5% w/w to about 3%, or about 1% w/w to about 2% of the composition.

The silicone-free antiperspirant and/or deodorant composition may further comprise a malodor reduction agent that masks, captures, neutralizes and/or prevents release of malodorous compounds. The present invention encompasses the use of any malodor reduction compound suitable for application on the human skin and also compatible for use in antiperspirant and/or deodorant compositions. Examples of malodor reduction compounds include, but are not limited to, citronellyl methylcrotonate (e.g. Sinodor™ by Givaudan), and an odor or wetness absorber (e.g. aluminium starch octenyl succinate such as Dry-Flo™). In embodiments, the malodor reduction agent(s) may be present in about 0.05% w/w to about 3% w/w, or about 1% w/w to about 2% w/w of the composition.

The antiperspirant and/or deodorant composition may further comprise hydrocarbon or synthetic polymer(s) that are suitable for application on the human skin and also compatible for use in antiperspirant and/or deodorant compositions. In embodiments the hydrocarbon or synthetic polymer is selected from diheptyl succinate polymer, capryloyl glycerin sebacic acid polymer, C₁₃-C₁₆ isoparaffin and heptyl undecylenate.

The antiperspirant and/or deodorant composition may further comprise natural oil(s) that are suitable for application on the human skin and also compatible for use in antiperspirant and/or deodorant compositions, such as a natural oil. In embodiments the natural oil is selected from soybean oil, olive oil, jojoba oil, castor oil, coconut oil, sunflower oil, safflower oil.

The antiperspirant and/or deodorant composition may further comprise a fatty alcohol suitable for application on the human skin and also compatible for use in antiperspirant and/or deodorant compositions. Examples include, but are not limited to, cetyl alcohol, cetearyl alcohol (including, but not limited to, C₁₆-C₁₈ cetearyl alcohol), and stearyl alcohol (including, but not limited to, C₁₈ stearyl alcohol).

The antiperspirant and/or deodorant composition may further comprise at least one humectant/moisturizer. Humectant(s)/moisturizer(s) may be particularly be useful in the manufacture of a cream. Examples include, but are not limited to, glycerin (e.g., Emery™ 917 Glycerine 99.7%). In embodiments, the humectant(s)/moisturizer(s) may be present in about 2% w/w to about 5% w/w, or about 2.5% w/w to about 3.5% w/w, or to about 3% w/w, about of the composition.

### Silicone-Free Antiperspirant Compositions Comprising Selected Emollient(s)

According to another aspect, the invention relates to the use of selected emollients in the manufacture of silicone-free antiperspirant and/or deodorant compositions.

Accordingly, there is provided a silicone-free antiperspirant and/or deodorant composition comprising at least one emollient selected from isoamyl laurate, C₁₂-C₁₅ alkyl benzoate, isopropyl myristate, dicaprylyl carbonate, oleyl erucate, hydrogenated ethylhexyl olivate, hydrogenated olive oil unsaponifiables, C₁₅-C₁₉ alkane and mixtures thereof.

In embodiments, the emollient is selected from isoamyl laurate, C₁₂-C₁₅ alkyl benzoate, isopropyl myristate, dicaprylyl carbonate, oleyl erucate, hydrogenated ethylhexyl olivate, hydrogenated olive oil unsaponifiables, C₁₅-C₁₉ alkane and mixtures thereof. In embodiments, the natural or synthetic wax(es) is(are) selected from jojoba esters, hydrogenated castor oil, beeswax, coconut wax, soy wax, ozokerite, ceresin and mixtures thereof.

The silicone-free antiperspirant and/or deodorant composition with the selected emollient(s) may also comprise at least one structuring agent, as defined herein before, as well as other ingredients including, but not limited to, natural or a synthetic wax(es), suspension agent(s), wetness absorber(s), antiperspirant agent(s), fragrance, essential oil(s), antioxidant(s), antimicrobial agent(s), soothing agent(s), anti-inflammatory agent(s), aesthetic agent(s), malodor reduction agent(s), thickener(s), emulsifier(s), humectant(s)/moisturizer(s), additive(s), etc. as defined herein before. Alternate compounds and desirable amounts in the composition are as provided hereinbefore. Proportions (i.e. % w/w amounts) are as defined hereinbefore.

### Silicone-Free Antiperspirant Compositions Comprising Selected Waxes

According to another aspect, the invention relates to the use of selected natural and/or synthetic waxes in the manufacture of silicone-free antiperspirant and/or deodorant compositions.

In embodiments, the natural or synthetic wax selected from jojoba esters, hydrogenated castor oil, beeswax, coconut wax, soy wax, ozokerite, ceresin and mixtures thereof.

In embodiments, the silicone-free antiperspirant and/or deodorant composition comprises the selected natural or synthetic wax(es) and at least one emollient selected from isoamyl laurate, C₁₂-C₁₅ alkyl benzoate, isopropyl myristate, dicaprylyl carbonate, oleyl erucate, hydrogenated ethylhexyl olivate, hydrogenated olive oil unsaponifiables, C₁₅-C₁₉ alkane and mixtures thereof.

The silicone-free antiperspirant and/or deodorant composition comprising the selected natural or synthetic wax(es) may also comprise at least one structuring agent, as defined herein before, as well as other ingredients including, but not limited to, suspension agent(s), wetness absorber(s), antiperspirant agent(s), fragrance, essential oil(s), antioxidant(s), antimicrobial agent(s), soothing agent(s), anti-inflammatory agent(s), aesthetic agent(s), malodor reduction agent(s), thickener(s), emulsifier(s), humectant(s)/moisturizer(s), additive(s), etc. as defined herein before. Alternate compounds and desirable amounts in the composition are as provided hereinbefore. Proportions (i.e. % w/w amounts) are as defined hereinbefore.

### Personal Hygiene Products

According to embodiments the antiperspirant and/or deodorant composition is used in the manufacture of various personal hygiene products, including but not limited to, creams, gels, soft solid sticks, body sprays, aerosols and the like. In preferred embodiments, the antiperspirant and/or deodorant composition is molded as a solid stick.

An antiperspirant and/or deodorant composition, and a personal hygiene product according to the invention may be particularly useful in controlling malodors and/or transpiration in a situs comprising a malodor and/or a situs that will become malodorous (e.g. underarm, feet, hands, and/or other intimates area (e.g. under breast) etc.). Antiperspirants are typically applied to the underarms, while deodorants may be used underarms and also on feet and other areas in the form of body sprays.

The antiperspirant and/or deodorant composition of the invention may be shaped as granules, a powder, a bar, a tablet, a cream, a gel, etc. In embodiments, the antiperspirant and/or deodorant composition is molded as a soft-solid stick. As used herein the term “soft-solid stick” refers to a stick having a hardness of about 40 mm/10 to about 90 mm/10, as measured according to ASTM D5 standards (e.g. 26.1-27.1° C., Needle H1280, 2.5 g).

In embodiments, the antiperspirant and/or deodorant composition is adapted for emulsion/dispersion type systems. In embodiments, the antiperspirant and/or deodorant composition is commercialized in the form of a soft-solid stick, as an antiperspirant cream or as a gel.

### Methods of Manufacture

Another aspect of the present invention concerns methods of manufacturing silicone-free antiperspirant and/or deodorant compositions as defined herein.

In one embodiment, the method of making a silicone-free antiperspirant and/or deodorant comprises preparing a antiperspirant and/or deodorant composition comprising at least one emollient selected from isoamyl laurate, C₁₂-C₁₅ alkyl benzoate, isopropyl myristate, dicaprylyl carbonate, oleyl erucate, hydrogenated ethylhexyl olivate, hydrogenated olive oil unsaponifiables, C₁₅-C₁₉ alkane and mixtures thereof.

In one embodiment, the method of making a silicone-free antiperspirant and/or deodorant comprises preparing a antiperspirant and/or deodorant composition comprising isoamyl laurate and at least one additional emollient selected from C₁₂-C₁₅ alkyl benzoate, isopropyl myristate, dicaprylyl carbonate, oleyl erucate, hydrogenated ethylhexyl olivate, hydrogenated olive oil unsaponifiables, C₁₅-C₁₉ alkane and mixtures thereof.

In another embodiment, the method of making a silicone-free antiperspirant and/or deodorant composition comprises preparing a composition comprising a mixture of isoamyl laurate (preferably in combination with one or more of emollients such as C₁₂-C₁₅ alkyl benzoate, isopropyl myristate, dicaprylyl carbonate, oleyl erucate, hydrogenated ethylhexyl olivate, hydrogenated olive oil unsaponifiables, C₁₅-C₁₉ alkane and mixtures thereof) and a natural or synthetic wax.

In another embodiment, the method of making a silicone-free antiperspirant composition comprises steps of: (a) heating and mixing at least one emollient, at least one structuring agent and at least one natural or synthetic wax until they are completely melted and dispersed into a liquid mixture; (b) cooling the liquid mixture while maintaining the mixture in a liquid form; (c) adding and mixing at least one antiperspirant agent, at least one suspension agent and at least one wetness absorber to the cooled liquid mixture; (d) optionally adding one or more additive(s) such as an antimicrobial agent, an antioxidant, a soothing agent, an odor fighting agent, etc. at a temperature preserving integrity of the additive(s); (e) homogenizing to ensure a proper suspension and dispersion of all ingredients; and (f) optionally adding and mixing a fragrance; thereby obtaining a silicone-free antiperspirant composition.

Preferably, the wax(es) and structuring agent(s) are added separately in order to ensure proper melting and mixing into the emollient(s). In embodiments, at least one structuring agent is added before the natural and/or synthetic wax(es). In alternative, the wax(es) could be added before the structuring agent(s), depending on their respective fusion temperature and/volume.

Preferably, the above are mixed to obtain the following ratios: a ratio of about 48:6 (% w/w) for the emollient(s):wax(es) to properly melt the wax(es) in the emollient(s); a ratio of about 48:6:19.7 (% w/w) for the emollient(s):wax(es):structuring agent(s) to obtain an acceptable hardness for a soft-solid stick.

According to one embodiment, the method is directed to the manufacture of a silicone-free antiperspirant composition, and it comprises the steps of:
1) premixing at least one emollient under heating;
2) successively adding at least one structuring agent and at least one wax under mixing until completely melted and dispersed;
3) cooling the mixture of step 2) while maintaining a liquid mixture;
4) adding and mixing at least one antiperspirant agent and suspension agent and at least one absorption agent to the cooled liquid mixture of step 3);
5) optionally adding one or more additive selected from an antimicrobial agent, an antioxidant, a soothing agent, an odor fighting agent, etc. at a temperature preserving integrity of the additive(s);
6) homogenizing to ensure a proper suspension and dispersion of all ingredients;
7) optionally adding and mixing a fragrance.
For obtaining of a soft solid stick, the method further comprises the steps of:
8) pouring the liquid mixture into stick molds; and
9) refrigerating molded sticks for solidification.

In embodiments, the at least one emollient is selected from the group consisting of isoamyl laurate, C₁₂-C₁₅ alkyl benzoate, isopropyl myristate, dicaprylyl carbonate, oleyl erucate, hydrogenated ethylhexyl olivate, hydrogenated olive oil unsaponifiables, C₁₅-C₁₉ alkane and mixtures thereof. Preferably, at least one emollient comprises isoamyl laurate.

In embodiments, at least one wax is a natural or synthetic wax, as defined hereinbefore.

In embodiments, at least one structuring agent is as defined hereinbefore.

In embodiments, the suspension agent is as defined hereinbefore.

In embodiments, the ingredients are added and mixed in proportions (i.e. % w/w amounts) as defined hereinbefore.

Those skilled in the art will recognize, or be able to ascertain, using no more than routine experimentation, numerous equivalents to the specific procedures, embodiments, claims and examples described herein. Such equivalents are considered to be within the scope of this invention and covered by the claims appended hereto. The invention is further illustrated by the following example, which should not be construed as further or specifically limiting.

### EXAMPLES

### Example 1

### Manufacture of Antiperspirant Sticks

A first antiperspirant stick having the following formulation was prepared:

| | | % (w/w) |
|---|---|---|
| TRADE NAME | | IN THE |
| (INCI NAME) | FUNCTION(S) | FORMULATION |
| Qualix D5-R77 ™ | Emollient | 21.85 |
| (Isoamyl Laurate) | | |
| Finsolv TN ™ | Emollient | 6 |
| (C₁₂-C₁₅ alkyl benzoate) | | |
| Tegosoft OER MB ™ | Emollient | 6 |
| (Oleyl Erucate + antioxydants) | | |
| Fision EcoSil ™ | Emollient | 14 |
| (Hydrogenated Ethylhexyl Olivate, | | |
| Hydrogenated Olive Oil | | |
| Unsaponifiables + antioxidant) | | |
| Lanette 18 Deo ™ | Structuring | 18.5 |
| (Stearyl Alcohol) | agent | |
| Kalcol 6098 ™ | Structuring | 1.2 |
| (Stearyl Alcohol + Cetyl | agent | |
| Alcohol + Myristyl Alcohol) | | |
| Floraesters 70 ™ | Natural | 1 |
| (jojoba esters) | vegetal wax | |
| Castorwax MP-80 ™ | Natural | 5 |
| (Hydrogenated Castor Oil) | vegetal wax | |
| Achieve DA 5390-04 ™ | Antiperspirant | 20.4 |
| (Aluminum Zirconium | agent (salt) | |
| Trichlorohydrex Glycine + water) | | |
| Cabosil M5P ™ | suspension | 2.5 |
| (silica) | agent | |
| Dry-Flo Pure ™ (aluminum Starch | Wetness | 2.5 |
| Octenylsuccinate + water) | absorber | |
| D-Alpha Tocopheryl Acetate USP | Antioxidant | 0.05 |
| | (natural) | |
| Fragrance | Fragrance | 1 |

A second antiperspirant stick, similar to the first one, having the following formulation was prepared:

| | | % (w/w) |
|---|---|---|
| TRADE NAME | | IN THE |
| (INCI NAME) | FUNCTION(S) | FORMULATION |
| Qualix D5-R77 ™ | Emollient | 21.85 |
| (Isoamyl Laurate) | | |
| Finsolv TN ™ | Emollient | 6 |
| (C₁₂-C₁₅ alkyl benzoate) | | |
| Tegosoft OER MB ™ | Emollient | 6 |
| (Oleyl Erucate + antioxydants) | | |
| Emosmart L19 ™ | Emollient | 14 |
| (C₁₅-C₁₉ alkane) | | |
| Lanette 18 Deo ™ | Structuring | 18.5 |
| (Stearyl Alcohol) | agent | |
| Kalcol 6098 ™ | Structuring | 1.2 |
| (Stearyl Alcohol + Cetyl | agent | |
| Alcohol + Myristyl Alcohol) | | |
| Floraesters 70 ™ | Natural vegetal | 1 |
| (Jojoba Esters) | wax | |
| Castorwax MP-80 ™ | Natural vegetal | 5 |
| (Hydrogenated Castor Oil) | wax | |
| Achieve DA 5390-04 ™ | Antiperspirant | 20.4 |
| (Aluminum Zirconium | agent (salt) | |
| Trichlorohydrex Glycine + water) | | |
| Cabosil M5P ™ | suspension | 2.5 |
| (silica) | agent | |
| Dry-Flo Pure ™ (aluminum Starch | Wetness | 2.5 |
| Octenylsuccinate + water) | absorber | |
| Vitamin E Acetate Care ™ | Antioxidant | 0.05 |
| | (synthetic) | |
| Fragrance | Fragrance | 1 |

A third antiperspirant stick, similar to the first and second ones, having the following formulation was prepared:

| | | % (w/w) |
|---|---|---|
| TRADE NAME | | IN THE |
| (INCI NAME) | FUNCTION(S) | FORMULATION |
| Qualix D5-R77 ™ | Emollient | 21.85 |
| (Isoamyl Laurate) | | |
| Finsolv TN ™ | Emollient | 6 |
| (C₁₂-C₁₅ alkyl benzoate) | | |
| Tegosoft OER MB ™ | Emollient | 6 |
| (Oleyl Erucate + antioxydants) | | |
| Fision EcoSil ™ | Emollient | 14 |
| (Hydrogenated Ethylhexyl Olivate, | | |
| Hydrogenated Olive Oil | | |
| Unsaponifiables + antioxidant) | | |
| Ecorol ™ | Structuring | 18.5 |
| (Stearyl Alcohol) | agent | |
| Kalcol 6098 ™ | Structuring | 1.2 |
| (Stearyl Alcohol + Cetyl | agent | |
| Alcohol + Myristyl Alcohol) | | |
| Floraesters 70 ™ | Natural vegetal | 1 |
| (Jojoba Esters) | wax | |
| Castoroil MP-80 | Natural vegetal | 5 |
| (Hydrogenated Castor Oil) | wax | |
| Achieve DA 5390-04 ™ | Antiperspirant | 20.4 |
| (Aluminum Zirconium | agent (salt) | |
| Trichlorohydrex Glycine + water) | | |
| Cabosil M5P ™ | suspension | 2.5 |
| (silica) | agent | |
| Dry-Flo Pure ™ (aluminum Starch | Wetness | 2.5 |
| Octenylsuccinate + water) | absorber | |
| Vitamin E Acetate Care ™ | Antioxidant | 0.05 |
| | (synthetic) | |
| Fragrance | Fragrance | 1 |

For each of the three formulations above, the ingredients were combined together according to the following procedure to make a soft-solid stick:
1) premixing, in accordance to the desired formula, the isoamyl laurate (e.g., Qualix D5-R77™), the C₁₂-C₁₅ alkyl benzoate (e.g., Finsolv TN™), the Oleyl Erucate (e.g., Tegosoft OER MB™), the C₁₅-C₁₉ Alkane (e.g., Emosmart L19™) and/or the hydrogenated ethylhexyl olivate, hydrogenated olive oil unsaponifiables (e.g., Fision EcoSil™) under heating;
2) successively adding the structuring agent (e.g., Lanette 18 Deo™+Kalcol 6098™; or Ecorol™+Kalcol 6098™) and the natural waxes (e.g., Floraesters 70™+Castorwax MP80™; or Floraesters 70™+Castoroil MP-80™) under mixing until complete mixing and dispersion;
3) cooling the mixture of step 2) while maintaining a liquid mixture;
4) adding and mixing to the cooled mixture of step 3) the antiperspirant agent (e.g., Achieve DA 5390-04™), the suspension agent (e.g., Cabosil M5P™) the wetness absorber (e.g., Dry-Flo Pure™) and the antioxidant (e.g., D-Alpha Tocopheryl Acetate USP or Vitamin E Acetate Care™);
5) homogenizing to ensure a proper suspension and dispersion of all the above-mentioned ingredients;
6) adding the fragrance into the liquid mix;
7) pouring the liquid mixture into stick molds; and
8) refrigerating molded sticks for solidification.

The three resultant soft-solid sticks exhibited desirable characteristics and properties, such as an opaque to off-white solid appearance, a smooth texture with no roughness or grittiness and a pleasant and comfortable spreadability. These properties are comparable, and even better, to existing silicone-containing sticks. Accordingly, the silicone-free sticks, in accordance with the present invention, possess desirable properties expected by a consumer, without the presence of undesirable silicone(s).

### Example 2

### Manufacture of an Antiperspirant Cream

An antiperspirant cream having the following formulation was prepared:

| | | % (w/w) |
|---|---|---|
| TRADE NAME | | IN THE |
| (INCI NAME) | FUNCTION(S) | FORMULATION |
| Water | Solvent | 44.1 |
| (Purified Water) | | |
| Emery 917 Glycerine 99.7% | Humectant/ | 3 |
| (Glycerin) | Moisturizer | |
| FiberDesign Sensation ™ | Biobased | 1 |
| (Citrus Limon (Lemon) Peel | Thickener/-gelifying | |
| Powder, Sclerotium Gum) | agent/texturizer/ | |
| | emulsion stabilizer | |
| Qualix D5-R77 ™ | Emollient | 12.45 |
| (Isoamyl Laurate) | | |
| Finsolv TN ™ | Emollient | 2 |
| (C₁₂-C₁₅ alkyl benzoate) | | |
| Tegosoft OER MB ™ (Oleyl | Emollient | 2 |
| Erucate + antioxydants) | | |
| Florasun 90 ™ | Emollient | 3.9 |
| (Helianthus Annuss | | |
| (Sunflower) Seed Oil) | | |
| Floraesters 70 ™ | Natural vegetal wax | 1.7 |
| (Jojoba Esters) | | |
| Ecorol18 Deo ™ | Structuring agent | 3 |
| (Stearyl Alcohol) | | |
| Emulgin B2 ™ | Emulsifier | 3 |
| (Ceteareth-20) | | |
| Vitamin E Acetate Care | Antioxidant | 0.05 |
| (Tocopheryl Acetate) | (Synthetic) | |
| Tapioca Pure 28 ™ | Wetness absorber | 1.4 |
| (Tapioca Starch) | | |
| Achieve DA 5390-04 ™ | Antiperspirant | 19 |
| (Aluminum Zirconium | agent (salt) | |
| Trichlorohydrex | | |
| Glycine + water) | | |
| Tazman Pepper AF ™ | Anti-inflammatory | 1 |
| (Glycerin, Water, Tasmania | agent | |
| Lanceolata Fruit/Leaf Extract) | | |
| Euxyl PE 9010 ™ | Antimicrobial | 0.9 |
| (Phenoxyethanol, | agent/Preservative | |
| Ethylhexylglycerin) | | |
| Fragrance | Fragrance | 1.5 |

For this formulation, the ingredients were combined together according to the following procedure to make an antiperspirant cream:
1) in a first vessel, premixing water and glycerin (e.g., Emery 917 Glycerine 99.7%) under heating;
2) slowly adding FiberDesign Sensation™ until complete mixing and dispersion;
3) in a second vessel, premixing Qualix D5-R77™, Finsolv TN™, Tegosoft OER™, Florasun 90™, Floraester 70™, Ecorol 18 Deo™, Emulgin B2™ and Vitamin E acetate under heating;
4) adding the premix from the second vessel (step 3) to the premix of the first vessel (step 2) under heating and mixing;
5) adding tapioca starch under mixing and homogenizing to ensure a complete emulsion;
6) cooling the mixture/emulsion of step 5;
7) adding the antiperspirant agent Achieve DA 5390-04™ (solubilized beforehand in water);
8) adding the active ingredient (Tazman Pepper™), the preservative (Euxyl PE 9010™) and the fragrance into the mixture of step 7;
9) cooling the mixture under mixing until it reaches room temperature, thereby obtaining an antiperspirant cream.

The resulting antiperspirant cream exhibited desirable characteristics and properties, such as an opaque cream appearance, a smooth texture and a pleasant and comfortable spreadability. These properties are comparable, and even better, to existing silicone-containing antiperspirant creams. Accordingly, the silicone-free antiperspirant cream of the invention possesses desirable properties expected by a consumer, without the presence of undesirable silicone(s).

### Example 3

### Stability Testing

Tests were carried out to evaluate shelf-life stability of silicone-free antiperspirant sticks in accordance with the present invention.

Briefly, silicone-free antiperspirant sticks manufactured in accordance with Example 1 (i.e. third antiperspirant stick). Samples were tested at 25° C./60% RH and at 40° C.|75% RH for 26 weeks. No significant variations were noted during the study.

In addition, a water activity analysis was performed and the measured value was found to be less than 0.25. Based on this minimal water activity, the formula was considered hostile to bacterial growth and no additional antimicrobial effectiveness test (AET) was required.

In conclusion, based on the stability data obtained from this accelerated stability study, the expected viable shelf life of the silicone-free antiperspirant sticks is extrapolated to be at least 24 months.

### Example 4

### Irritability Testing in Human Subjects

Potential dermal irritability of a silicone-free antiperspirant composition in accordance with the present invention was evaluated (i.e. third antiperspirant stick of Example 1).

The objective of the study was to confirm absence of potential primary irritability potential, accumulated dermal irritability and dermal sensitization of the silicone-free product for topical uses in maximized conditions, application area and controlled quantity. The study was planned and conducted under regulatory guidelines and standards for research involving humans and approved by a committee of ethics in clinical research.

Briefly, 55 study participants aged between 18 and 60 years completed the study. There were no reports of evidence of adverse reaction during the study. The product test and a control were applied on filter paper discs of a patch test and then applied on the hack (shoulder area) right or left at research participants. Two (2) contact tests were applied, each of which corresponds to required ratings, primary irritant and irritant cumulative dermal. For evaluation of primary irritability, the area was evaluated after 48 and 96 hours of the first application of the investigational product. For assessment of accumulated dermal irritability, every 48 hours the study participants returned for patch removal and site readings after 30 minutes of rest. Not having any change, soon after reading the contact test was reapplied. This procedure was repeated during the 3-week period. The applications were performed three times a week for three consecutive weeks, every other day, making a total of nine applications. Survey participants were evaluated by medical dermatologist at the beginning and end of the study and followed throughout the course of the same.

The tested product did not induce any irritating phenomena. It did not cause either significant or detectable skin sensitization during the study period. The product is thus considered safe tor topical use, supporting in this way the qualifier “dermatologically tested”.

### Example 5

### Antiperspirant Efficacy

Antiperspirant effectiveness of a silicone-free antiperspirant composition in accordance with the present invention (i.e., third antiperspirant stick of Example 1) was assessed in human subjects.

Briefly, a study was carried out in accordance with the guidelines for effectiveness testing of OTC Antiperspirant Drug Products (FDA). Twenty-two (22) research participants completed the study and their mean age was 50±9 years. There were no reports or evidence of adverse reaction during the study. The methodology consisted to discontinue use of any products underarm, beyond those supplied by Laboratory for a period of 17 days. During the conditioning period, the participants were instructed to wash their underarms only with a mild soap dispensed by the laboratory. After the conditioning period, the initial evaluation was carried out in a hot room to verify the amount of sweat produced per armpit. To be approved it was necessary that the participant produced at least 100 mg of sweating per armpit for two consecutive sessions (T1 and T2) of 20 minutes. The approved participants received four (4) controlled applications of the investigational product in one armpit, while the other armpit received applications of the placebo. After the applications of the research product and placebo, the research participants were instructed not to wet, wash or pass any product in the armpits for the next 48 hours. After 48 hours of the last application of the investigational product and placebo, the participants were submitted to heat exposure in a hot room.

According to the study protocol and procedures that were used, it was concluded that the investigational product has antiperspirant effect for up to 48 hours. The investigational product is extra effective for up to 48 hours, i.e. it can reduce perspiration for at least 30% on a 48 h period.

### CONCLUSIONS

These examples clearly demonstrate that it is possible, according to the present invention, to formulate and obtain various antiperspirants and deodorants compositions that are free of silicone, and that are safe, stable and effective.

The silicone-free compositions and personal hygiene products according to the present invention also possess the benefit of being environmentally friendly because of the use of biodegradable and natural raw materials, greener choice of ingredients and reduction of wastes in the manufacturing process.

The silicone-free compositions and personal hygiene products according to the invention are advantageously well tolerated by the skin, they have a nice skin feel and aesthetic, they are stable and they are not expensive to manufacture. They also have a long-lasting antiperspirant effect.

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise. Thus, for example, reference to “an agent” includes one or more of such agents and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties and so forth used in the specification and claims are to be understood as being modified in all instances by the term “about”. At the very least, each numerical parameter should be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1-3. Canceled.

4. A silicone-free antiperspirant and/or deodorant composition comprising a plurality of emollients, wherein said plurality of emollients comprises isoamyl laurate and at least one additional emollient selected from the group consisting of C₁₂-C₁₅ alkyl benzoate, isopropyl myristate, oleyl erucate, hydrogenated ethylhexyl olivate, hydrogenated olive oil unsaponifiables, C₁₅-C₁₉ alkane and mixtures thereof.

5-6. (canceled)

7. The silicone-free composition of claim 4, further comprising at least one structuring agent and at least one natural or synthetic wax.

8-11. (canceled)

12. The silicone-free composition of claim 7, wherein the structuring agent is selected from the group consisting of stearyl alcohol, cetyl alcohol, myristyl alcohol and mixtures thereof.

13. The silicone-free composition of claim 4, further comprising a suspension agent selected from the group consisting of silica, cellulose powder and mixtures thereof.

14. (canceled)

15. The silicone-free composition of claim 4, further comprising a wetness absorber selected from the group consisting of aluminum starch, octenyl succinate, tapioca starch, potato starch, rice starch, corn starch, arrowroot powder, clays, hectorite, bentonite, zeolite and mixtures thereof.

16. (canceled)

17. The silicone-free composition of claim 4, wherein said composition consists of an antiperspirant composition further comprising an antiperspirant agent.

18. The silicone-free composition of claim 17, wherein said antiperspirant agent is selected from the group consisting of aluminium zirconium trichlorohydrex Gly, aluminium zirconium tetrachlorohydrex Gly, aluminum zirconium pentachlorohydrex Gly, aluminum zirconium octachlorohydrex Gly, aluminium chlorohydrate and aluminum sesquichlorohydrate.

19. The silicone-free composition of claim 4, further comprising a malodor reduction agent that masks, captures, neutralizes and/or prevents release of malodorous compounds.

20. The silicone-free composition of claim 19, wherein said malodor reduction agent is selected from the group consisting of citronellyl methylcrotonate, an odor absorber and mixtures thereof.

21. The silicone-free composition of claim 4, further comprising at least one of a fragrance and an essential oil.

22. The silicone-free composition of claim 4, further comprising an antioxidant selected from the group consisting of tocopherols, tocopheryl acetate, Pentaerythrityl Tetra-Di-t-Butyl Hydroxyhydrocinnamate and mixtures thereof.

23. (canceled)

24. The silicone-free composition of claim 4, further comprising at least one additive selected from the group consisting of an odor fighter, a soothing agent, a natural oil, an aesthetical agent, an antimicrobial agent and an anti-inflammatory agent.

25. The silicone-free composition of claim 4, comprising at least one of the following:
about 35% w/w to about 55% w/w of emollient(s);
about 16% w/w to about 20% w/w of structuring agent(s);
about 4% w/w to about 6% w/w of natural wax(es);
about 12% w/w to about 20% w/w of antiperspirant agent(s);
about 1.5% w/w to about 3% w/w of suspension agent(s);
about 0.05% w/w to about 0.5% w/w of antioxidant(s);
about 0% w/w to about 3% w/w of additive(s);
about 1.5% w/w to about 3% w/w wetness absorber; and
about 0% w/w to about 1.5% w/w fragrances.

26. The silicone-free composition of claim 4, wherein said silicone-free composition is anhydrous.

27. The silicone-free composition of claim 4, wherein said silicone-free composition is molded as a soft-solid stick or is in the form of a cream.

28. The silicone-free composition of claim 4, wherein said silicone-free composition is adapted for an application under the underarms, under the feet, in the hands, intimate areas and/or under the breast.

29-42. (canceled)

43. A silicone-free antiperspirant composition comprising:
about 2% w/w to about 5% w/w of humectant(s)/moisturizer(s);
about 0.05% w/w to about 1.5% w/w of thickener(s);
about 15% w/w to about 25% w/w of emollient(s);
about 1% w/w to about 3% w/w of natural wax(es);
about 2% w/w to about 4% w/w of structuring agent(s);
about 2% w/w to about 4% w/w of emulsifier(s);
about 0.01% w/w to about 0.1% w/w of antioxidant(s);
about 0.5% w/w to about 3% w/w wetness absorber(s);
about 12% w/w to about 25% w/w of antiperspirant agent(s);
about 0% w/w to about 3% w/w of additive(s);
about 0% w/w to about 2.5% w/w fragrance(s); and
solvent(s) to complete to 100% w/w.

44. The silicone-free antiperspirant composition of claims 43, comprising:
about 3% w/w of humectant(s)/moisturizer(s);
about 20.35% w/w of emollient(s);
about 1% w/w of thickener(s);
about 3% w/w of structuring agent(s);
about 1.7% w/w of natural wax(es);
about 19% w/w of antiperspirant agent(s);
about 0.05% w/w of antioxidant(s);
about 1.9% w/w of additive(s);
about 1.4% w/w wetness absorber(s);
about 1.5% w/w fragrance(s); and
about 44.1% w/w solvent.

45. The silicone-free antiperspirant composition of claim 43, wherein said silicone-free antiperspirant composition is formulated as a cream.

46-57. (canceled)

58. A personal hygiene product comprising a silicone-free composition as defined in claim 4.

59. The personal hygiene product of claim 58, wherein said product is at least one of a soft-solid stick and a cream.

60. An antiperspirant stick comprising a silicone-free composition as defined in claim 17.

61. The antiperspirant stick of claim 60, wherein the antiperspirant stick has a hardness of about 40 mm/10 to about 90 mm/10, as measured according to ASTM D5 standards at 26.1-27.1° C., Needle H1280, 2.5 g.

62. (canceled)

63. A method of controlling at least one of malodors and transpiration comprising: contacting a situs comprising a malodor and/or a situs that will become malodorous with a silicone-free composition as defined in claim 4.
